(19) United States
(12) Patent Application Publication
(10) Pub. No.: US 2025/0319441 A1
(43) Pub. Date: Oct. 16, 2025

(54) SYSTEMS AND METHODS FOR FILTRATION

(71) Applicant: BendBio, Inc., Sharon, MA (US)

(72) Inventor: Kyle Christopher Smith, Cambridge, MA (US)

(73) Assignee: BendBio, Inc., Sharon, MA (US)

(21) Appl. No.: 18/869,135

(22) PCT Filed: May 25, 2023
(86) PCT No.: PCT/US2023/023530

§ 371 Date: Nov. 25, 2024

Related U.S. Application Data
(60) Provisional application No. 63/346,701, filed on May 27, 2022.

(51) Int. Cl. B01D 63/08; B01D 63/10; B01D 69/02

(52) U.S. Cl. CPC B01D 63/088; B01D 63/10; B01D 69/02; B01D 2313/083; B01D 2325/0283

(57) ABSTRACT

In one aspect, the present disclosure generally relates to systems and methods for filtration. In some embodiments, filters are provided that include bypass channels, e.g., such that the filter is able to allow fluid flow to occur even if most or all of the filter elements are clogged. In some cases, the bypass channel may have a fluidic resistance that is higher than the filter elements, such that fluid preferentially passes through the filter elements. However, over time, as the filter elements become clogged with debris, the fluidic resistance of the filter elements may increase, e.g., such that it becomes greater than the bypass channel, and fluid may instead preferentially pass through the bypass channel. In contrast, in many prior art devices, once a filter has clogged, fluid can no longer flow through the filter.

### RELATED APPLICATIONS

This application claims the benefit of U.S. Provisional Patent Application Ser. No. 63/346,701, filed May 27, 2022, entitled “Systems and Methods for Filtration,” incorporated herein by reference in its entirety.

### FIELD

The present disclosure generally relates to systems and methods for filtration.

### BACKGROUND

Biological systems are intrinsically heterogeneous and dynamic and fluids derived from these systems vary widely in their characteristics. Such variability complicates and reduces the performance of clinical, biomanufacturing, and bioprocessing applications. Process efficiency and reliability may be improved by removing unnecessary biological fluid components (contaminants) before they become disruptive. Common contaminants include cellular debris, dead cells, cell aggregates, and clots. Filtration may be used to remove these contaminants and enhance downstream processing.

Many of the filters available are poorly suited for robust and consistent performance across highly variable biological samples. They feature simplistic designs (e.g., screens) that may be effective at trapping rigid, monodisperse particles but are ineffective at capturing the deformable, fibrous, and polydisperse debris particles that are common in biological samples. Such filters either rapidly accumulate debris and clog without full utilization of available membrane or allow small contaminants to freely pass through. The outcome depends on the characteristics of the sample being filtered.

### SUMMARY

The disclosure presented here addresses some of these limitations. By adding a bypass flow path, more filter regions become available to the fluid, and by applying the design principles described herein, the filter clogs in a progressive and predictable manner in some embodiments. In some embodiments, this may be implemented using conventional filter materials and fabrication techniques, or it may be implemented using microfluidic materials and fabrication techniques, etc.

Accordingly, the present disclosure generally relates to systems and methods for filtration. The subject matter of the present disclosure involves, in some cases, interrelated products, alternative solutions to a particular problem, and/or a plurality of different uses of one or more systems and/or articles.

In one aspect, the present disclosure generally relates to systems and methods for filtration. In some embodiments, filters are provided that include bypass channels, e.g., such that the filter is able to allow fluid flow to occur even if most or all of the filter elements are clogged. In some cases, the bypass channel may have a fluidic resistance that is higher than the filter elements, such that fluid preferentially passes through the filter elements. However, over time, as the filter elements become clogged with debris, the fluidic resistance of the filter elements may increase, e.g., such that it becomes greater than the bypass channel, and fluid may instead preferentially pass through the bypass channel. In contrast, in many prior art devices, once a filter has clogged, fluid can no longer flow through the filter.

Another aspect is generally directed to a device. In one set of embodiments, the device comprises a filter comprising a filter element and a bypass pathway positioned to flow a fluid around the filter element. In another set of embodiments, the device comprises a plurality of filter elements defining arms of a spiral, where the plurality of filter elements is positioned to define fluid channels between the arms of the spiral.

Yet another aspect is generally directed to a method. In one set of embodiments, the method comprises providing a device comprising a filter element and a bypass pathway positioned to flow a fluid around the filter element, flowing a fluid containing debris through the filter element such that at least some of the debris becomes entrapped in the filter element, and subsequently, flowing at least some of the fluid in the bypass pathway around the filter element.

In another set of embodiments, the method comprises flowing debris through a filter until 80% of the filter is internally filled with debris.

In some aspects, the present disclosure encompasses methods of making one or more of the embodiments described herein, for example, a filtration system. In still another aspect, the present disclosure encompasses methods of using one or more of the embodiments described herein, for example, a filtration system.

Other advantages and novel features of the present disclosure will become apparent from the following detailed description of various non-limiting embodiments of the disclosure when considered in conjunction with the accompanying figures.

### DETAILED DESCRIPTION

The present disclosure generally relates to systems and methods for filtration. Various non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In addition, it will be understood that these figures are merely examples, and that other embodiments of the present disclosure are also described in more detail herein.

One aspect is generally directed to a filter. In some embodiments, the filter may include a bypass pathway and a plurality of filter elements. As the filter elements get clogged or inactivated, for example, with debris, fluid may continue to flow through other, unclogged filter elements, and/or through the bypass pathway. The debris may include, for example, cells or cell lysate, proteins, DNA or RNA, lipids, precipitants, impurities, contaminants, bacteria, fungi, or the like. In some cases, the debris may be uncharacterized. Accordingly, the filter may be able to function even if most or all of the filter elements are clogged. For instance, even if a filter is fully clogged, fluid can still flow through or around the filter. In contrast, in many prior art devices, once a filter has clogged, fluid can no longer flow through the filter. This can result in an inoperable device that uses the filter, as fluid cannot flow past the filter into other parts of the device; often, such prior art devices will need to be taken out of service until the filter can be unclogged or a new filter installed. In contrast, even if a filter as discussed herein is fully clogged, e.g., with debris, the device containing the filter may still be able to perform, as fluid can still flow past the filter, e.g., via the bypass pathway. In some embodiments, the filter may have a generally spiral shape, although this is not a requirement. Non-limiting examples of generally spiral filters are shown in the figures and are discussed in more detail herein.

In some cases, the fluidic resistance of the bypass pathway may be greater than the fluidic resistance through the plurality of filter elements. Thus, fluid preferentially flows through the plurality of filter elements (although some fluid may still flow through the bypass pathway in certain cases). However, as portions of the filter elements become clogged or inoperable, e.g., due to debris, the fluidic resistance of those filter elements may increase, thus increasingly favoring flow through the bypass pathway. In some cases, fluid may thus flow through the filter even if the filter elements become clogged or inoperable during use, e.g., due to debris. For instance, fluid may flow through a filter even if at least 30%, at least 40%, at least 50%, at least 60%, at least 70%, at least 80%, at least 90%, at least 95%, or even 100% of the filter elements forming the filter become clogged or inoperable.

In some cases, the filter may be constructed and arranged to clog with debris in a progressive manner. For example, the filter elements or portions of filter elements near the inlet of the device may clog prior to filter elements or portions of filter elements near the outlet of the device. During use, the ratio of resistance of a portion of a filter element to its initial resistance (pre-use) may increase substantially as debris becomes trapped and impedes flow through the filter element. For example, during use the resistance a portion of filter element may increase at least 2-fold, at least 3-fold, at least 5-fold, at least 10-fold, at least 20-fold, at least 50-fold, at least 100-fold, etc. Given the progressive manner in which a filter may clog, the resistance increase in one portion of the filter may become significantly larger than the resistance increase in another portion of the filter. For example, the resistance increase of one portion of a filter element divided by the resistance increase of another portion of a filter element may be at least 2-fold, at least 3-fold, at least 5-fold, at least 10-fold, at least 20-fold, at least 50-fold, at least 100-fold, etc.

The changes in resistance that may occur during use may influence fluid flow through the filter device. As debris becomes trapped and impedes flow through a portion of filter element, the flow through an adjacent bypass pathway may increase as flow is diverted from the filter element pathway to the bypass pathway. For example, during use the flow through a portion of bypass pathway may increase at least 2-fold, at least 3-fold, at least 5-fold, at least 10-fold, at least 20-fold, at least 50-fold, at least 100-fold, etc. Given the progressive manner in which a filter may clog, the flow increase through a portion of bypass pathway in one portion of the filter may become significantly larger than the flow increase in another portion of the filter. For example, the flow increase of one portion of a bypass pathway divided by the flow increase of another portion of a bypass pathway may be at least 2-fold, at least 3-fold, at least 5-fold, at least 10-fold, at least 20-fold, at least 50-fold, at least 100-fold, etc.

The ratio of flow through an adjacent bypass pathway to flow through a portion of filter element may also increase during use in certain embodiments. For example, the ratio may increase at least 2-fold, at least 3-fold, at least 5-fold, at least 10-fold, at least 20-fold, at least 50-fold, at least 100-fold, etc. The flow ratio in one portion of the filter may become significantly larger than the flow ratio in another portion of the filter. For example, the flow ratio of one portion of a filter divided by the flow ratio of another portion of the filter element may increase at least 2-fold, at least 3-fold, at least 5-fold, at least 10-fold, at least 20-fold, at least 50-fold, at least 100-fold, etc.

The filter may have any suitable pore size. For example, the filter may have an average pore size of less than 100 mm, less than 75 mm, less than 50 mm, less than 25 mm, less than 10 mm, less than 5 mm, less than 3 mm, less than 1 mm, less than 750 micrometers, less than 500 micrometers, less than 250 micrometers, less than 200 micrometers, less than 150 micrometers, less than 100 micrometers, less than 75 micrometers, less than 50 micrometers, less than 25 micrometers, less than 10 micrometers, less than 5 micrometers, less than 3 micrometers, less than 2 micrometers, less than 1 micrometers, less than 0.5 micrometers, less than 0.3 micrometers, less than 0.1 micrometers, etc. Pore size may be determined via microscopic inspection, by passing spherical particles with known diameters through the filter and determining when 50% of the particles are able to pass through the pores, or by other techniques known to those of ordinary skill in the art.

In some cases, one or more of the filters may be rotationally symmetric. For instance, a filter may exhibit 2-fold, 3-fold, 4-fold, 5-fold, 6-fold, 7-fold, 8-fold, 10-fold, 12-fold, 16-fold, or more degrees of rotational symmetry, in various embodiments.

In some cases, one or more of the filters may be translationally symmetric. For instance, a filter may exhibit 2-fold, 3-fold, 4-fold, 5-fold, 6-fold, 7-fold, 8-fold, 10-fold, 12-fold, 16-fold, or more degrees of translational symmetry, in various embodiments.

In some embodiments, one or more filters such as described herein may be used. These may be fluidly connected, e.g., in series and/or in parallel. For example, if a first filter and a second filter are fluidly connected in series, then even if the first filter becomes clogged or inoperable, fluid may flow through the bypass pathway of the first filter to reach the second filter, which may still be in operation (for example, because most of the debris has been trapped in the first filter, thus resulting in less debris reaching the second filter and potentially clogging it). As another example, if a first filter and a second filter are fluidly connected in parallel, then even if one of the filters is clogged, then at least some of the fluid can flow through the second filter. If the second filter also becomes clogged, fluid can still flow through the device through bypass channels that may be present in the first filter and/or in the second filter. In addition, in some embodiments, if 3, 4, 5, 6, or more filters are present, the filters may be fluidly connected in any suitable configuration. For example, the filters may all be in series, may all be in parallel, or some may be in series with each other and some may be in parallel with each other, etc. In addition, if more than one filter is present, the filters may each independently have the same or different configurations.

In some embodiments, the bypass pathway may have a cross-sectional dimension substantially larger than a cross-sectional dimension within the filter elements, e.g., such that the bypass pathway is less likely to clog due to debris than the filter elements. For instance, the bypass pathway may have an average cross-sectional dimension that is at least 2 times, at least 3 times, at least 4 times, at least 5 times, at least 6 times, at least 7 times, at least 8 times, at least 10 times, at least 15 times, at least 20 times, at least 25 times, at least 30 times, at least 40 times, at least 50 times, etc. bigger than the average cross-sectional dimension within the filter elements. The bypass pathway also may have a fluid flow pathlength that is significantly longer than the fluid flow pathlength through the filter elements, e.g., such that the fluidic resistance of the bypass pathway may be greater than the fluidic resistance through the plurality of filter elements. For instance, the bypass pathway may have a length that is at least 2 times, at least 3 times, at least 4 times, at least 5 times, at least 6 times, at least 7 times, at least 8 times, at least 10 times, at least 15 times, at least 20 times, at least 25 times, at least 30 times, at least 40 times, at least 50 times, etc. longer than the fluid flow pathlength through the filter elements.

In some embodiments, the long axis of one or more filter elements extends from the inlet to the outlet with the space between the filter elements defining bypass pathways from the inlet to the outlet. The long axis of the filter elements may generally define an angle with respect to the shortest path from inlet to outlet through the filter elements, e.g., to create or determine the pressure drop across the filter element. As the angle increases in magnitude (toward 90°), the pressure drop per unit length across the filter element increases relative to the pressure drop per unit length along the bypass pathway, increasing the tendency of flow to pass through the filter element rather than through the bypass pathway. This angle may be at least 5°, at least 10°, at least 15°, at least 20°, at least 25°, at least 30°, at least 35°, at least 40°, at least 45°, at least 50°, at least 55°, at least 60°, at least 65°, at least 70°, at least 75°, at least 80°, at least 85°. In some embodiments this angle is fixed. In other embodiments the angle may vary with position.

In some embodiments, a single continuous bypass pathway may wrap around a filter element such that the bypass pathway is approximately twice the length of the long axis of the filter element. In such embodiments, the bypass flow on opposite sides of the filter element may be approximately opposite in direction. In other embodiments, two distinct bypass pathways may lie alongside a single filter element such that each bypass pathway is approximately equal in length to the long axis of the filter element. In such embodiments, the bypass flow on opposite sides of the filter element may be in approximately the same direction. In yet other embodiments, a single continuous bypass pathway may lie alongside both sides of a single filter element by virtue of both structures wrapping around a central axis (as in a spiral or helix). In such embodiments, the bypass pathway may be approximately equal in length to the long axis of the filter element, and the bypass flow on opposite sides of the filter element may be in approximately the same direction.

The filter elements and/or bypass pathways (either in their entirety or in cross-section) may be or at least approximate geometric shapes in some embodiments. For example, the filter elements may be or approximate spirals, helices, lines, arcs, splines, and/or other shapes. Similarly, the bypass pathways may approximate spirals, helices, lines, arcs, splines, and/or other shapes in certain embodiments. In some embodiments, the shapes of the filter elements and/or bypass pathways may combine multiple shapes to form more complex geometries.

As mentioned, in some embodiments, the filter may have a generally spiral shape, although this is not a requirement. Fluid may flow inwardly towards the center or outwardly towards the edges, depending on the embodiment. The filter may have any number of spiral arms present, e.g., 1, 2, 3, 4, 5, 6, 7, 8, 9, 10, 12, 14, 16, 18, 20, 25, 30, 32, 50, 64, 100, 128, or more, in various embodiments. In addition, if more than one filter is present, the filters may each independently have the same or different configurations.

In some embodiments, the filter may have a generally helical shape. Fluid may generally flow in the direction of the axis of the helix. The filter may have any number of helical arms present, e.g., 1, 2, 3, 4, 5, 6, 7, 8, 9, 10, 12, 14, 16, 18, 20, 25, 30, 32, 50, 64, 100, 128, or more, in various embodiments. In addition, if more than one filter is present, the filters may each independently have the same or different configurations.

Non-limiting examples of various filters are now described with reference to the accompanying figures. However, it should be understood that these are presented merely by way of example, not limitation, and that other embodiments of the present disclosure are also possible in addition to the ones described as follows. In the figures and as discussed herein, the term “filter region” should be understood to be equivalent to “filter element.”

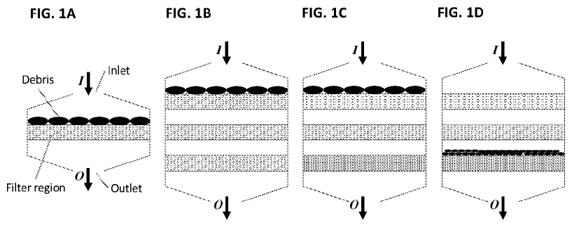

FIG. 1. (FIG. 1A) Conventional filters comprise one or more filter regions placed between an inlet and outlet. All flow must pass through the filter region(s). As debris accumulates on the filter, flow is impeded. When the filter becomes fully clogged, fluid may no longer flow from input to output. Conventional filters fail in a “closed”, rather than “open”, state. (FIG. 1B) Adding more filter regions in series with the first filter region does not increase the overall filter capacity because once the first filter region becomes clogged, the flow cannot access the filter regions further downstream. (FIG. 1C) and (FIG. 1D) One strategy for increasing capacity is the use of graded filter regions, in which each successive filter region has properties (e.g., pore size) that target smaller debris. Large debris is trapped in the upstream filter regions and small debris in the downstream filter regions. For consistent input samples with debris of varying size, this approach may increase the capacity of the filter by better utilizing all filter regions. However, the debris in biological samples tends to vary not just within a single sample but also across samples. As such, one sample may have large debris that clogs the first filter region, leaving the downstream filter regions unutilized, as in (FIG. 1C). Another sample may have smaller debris that clogs the last filter region, leaving the upstream filter regions unutilized, as in (FIG. 1D).

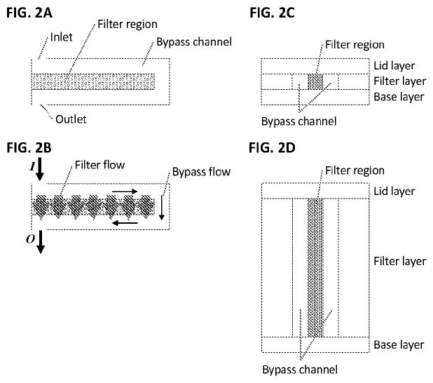

FIG. 2. (FIG. 2A) A filter with bypass (FwB) is one example embodiment described here. An FwB comprises a filter region placed between an inlet and outlet and a bypass channel that enables flow to pass from the inlet to the outlet without passing through a filter region, i.e., an FwB is arranged to allow flow around the filter region. (FIG. 2B) An FwB may be configured in some embodiments so that most flow passes through the filter region when the filter region is unclogged due to its being the lowest resistance (and shortest) path from the inlet to the outlet. The bypass channel may be constructed and arranged to be relatively longer and/or narrower, such that only a fraction of the total flow passes through the bypass. (FIG. 2C) and (FIG. 2D) In cross section, the FwB filter region and bypass channel (as shown in (FIG. 2A) lie within the filter layer in this example. The filter layer may be positioned between the base layer and the lid layer. FwB units may be stacked such that the lid layer of one FwB unit also serves as the base for another FwB unit. The FwB may be a microfluidic device with the filter region fabricated using microfabrication techniques, such as photolithography, soft lithography, casting, embossing, molding, or printing, etc. The FwB may also be formed using macrofluidic manufacturing techniques incorporating a pre-formed filter region, such as a track etched membrane, screen, or porous membrane. Other fabrication techniques are also possible, including but not limited to those described herein.

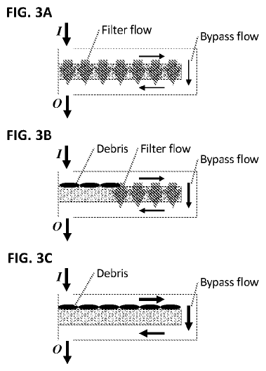

FIG. 3. (FIG. 3A) In this embodiment, when the filter region is free of debris, most flow passes through the filter region nearest the inlet and outlet. (FIG. 3B) As a result, debris may be initially captured on the filter region nearest the inlet and outlet, impeding further flow through this region. The flow then may follow a bypass path until reaching and then passing through an unclogged filter region. As a result, the filter region may clog progressively from the side nearest the inlet and outlet to the side farthest from the inlet and outlet. (FIG. 3C) Eventually, the filter region may become completely clogged with debris such that all flow follows the bypass channel. This bypass flow remains unfiltered, but flow from the inlet to the outlet is sustained. Unlike a conventional filter, the FwB fails in an “open” rather than in a “closed” state.

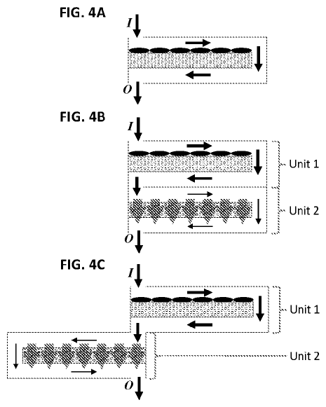

FIG. 4. (FIG. 4A) When the filter region is completely clogged with debris in this embodiment, the flow from the inlet to the outlet follows the bypass channel. (FIG. 4B) and (FIG. 4C) Because the filter region clogs progressively in this example, an FwB may integrate one or more filtration units in series. In this example, after Unit 1 fails and all flow passes through the Unit 1 bypass channel, it is directed to a second Unit 2, which is in the same state as Unit 1 prior to clogging. Unit 2 then gradually and progressively clogs. Depending on the capacity required for a given application, a plurality of units may be combined in series and/or parallel. In contrast to a multi-layer conventional filter, some or all of the filter regions in an FwB remain accessible to flow, thereby maximizing the overall filtration capacity.

Accordingly, it should be understood that in various embodiments, a plurality of filters, including those described herein, may be combined in any suitable arrangement, e.g., in series and/or in parallel. As a non-limiting example, in one embodiment, there may be 2, 3, 4, 5, 6, 7, 8, 9, 10, or more filters arranged in series. In addition, in other embodiments, one or more of these may be arranged in parallel, e.g., such that there are 2, 3, 4, 5, 6, 7, 8, 9, 10, or more filters arranged in parallel. Any combination of filters in series and/or parallel are also possible in yet other embodiments.

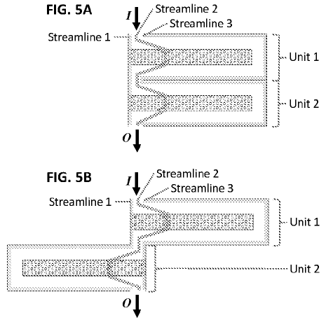

FIG. 5. (FIG. 5A) In this example, streamline 1 enters the FwB farthest from the bypass channel, Streamline 3 enters closest to the bypass channel, and Streamline 2 enters between Streamlines 1 and 2. While Streamlines 1 and 2 pass through the filter regions of Unit 1 and Unit 2, Streamline 3 passes through the bypass channels of Unit 1 and Unit 2. As such, the fluid in Streamline 3 remains unfiltered in this example. (FIG. 5B) In this example, the orientation of Unit 2 is flipped. Now, Streamline 1 passes through the filter region of Unit 1 and the bypass channel of Unit 2, Streamline 2 passes through the filter regions of Unit 1 and Unit 2, and Streamline 3 passes through the bypass channel of Unit 1 and the filter region of Unit 2. As such, all of the fluid may pass through as least one of the filter regions.

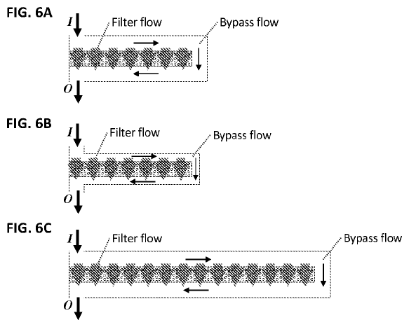

FIG. 6. (FIG. 6A) In certain embodiments, the fraction of fluid that passes through the bypass channel may depend in some cases on the fluidic resistance of the bypass channel relative to the filter region. There are various ways that the relative resistance may be altered. (FIG. 6B) For example, reducing the width of the bypass channel may increase its fluidic resistance in some cases. As a result, less of the total flow may pass through the bypass and more may pass through the filter region. (FIG. 6C) Similarly, in another embodiment, increasing the bypass channel length may increase its fluidic resistance and/or decrease the fluidic resistance of the filter region by increasing its width (normal to filter flow). Less of the total flow may pass through the bypass and more may pass through the filter region in this example. In some cases, the fluidic resistance of the filter region may depend on certain properties, such as but not limited to its overall thickness and the density and size of flow paths through it. Additionally, the fluidic resistance of the filter region may change (e.g., increase) as debris accumulates in it.

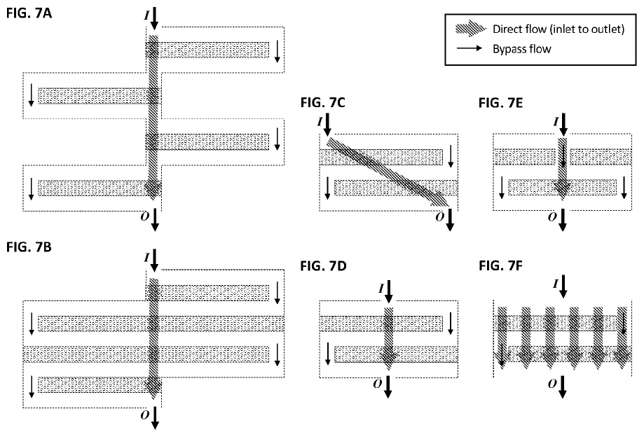

FIG. 7. Many FwB configurations are possible. Some configurations can allow for bypass flow from inlet to outlet that passes around, rather than through, the filter regions, in certain cases. In some embodiments, some or all of the filter regions may be accessible to flow, which may allow progressive clogging of the FwB in some cases. Prior to clogging, most flow may follow a predominantly direct path from inlet to outlet, as this path has the steepest drop in pressure. In contrast, the bypass flow may follow a more circuitous path that provides access to all filter regions and has a relatively higher resistance than the direct flow path. (FIG. 7A)-(FIG. 7F) show non-limiting example configurations. Many others are possible.

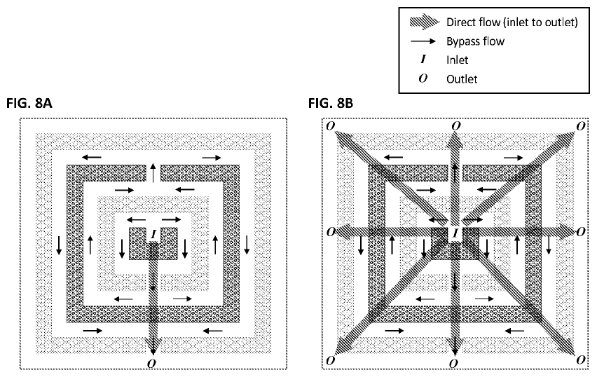

FIG. 8. FwBs may utilize non-linear/non-planar filter regions and multiple or distributed inlets and outlets in some embodiments. (FIG. 8A) A central inlet may (incompletely) be enclosed by filter regions that are “folded” around it in this example. There can be a direct flow path to the single outlet. The filter regions may collectively define circuitous bypass channels, which may allow bypass flow from the inlet to the outlet. Because the bypass channels are relatively long and indirect paths, fluid flow tends toward a more direct path from inlet to outlet, e.g., unless the interior filter regions become clogged. (FIG. 8B) In this example, a similar FwB configuration but with multiple outlets distributed around an outer region of the device is described. Collectively, these multiple outlets may constitute an outlet region. In this example configuration, the direct flow path from inlet to the outlet is divergent and less concentrated than the flow in the example of (FIG. 8A). Bypass flow paths may be present between the inlet and all outlets. Additionally, the inlets and outlets (and flow direction) may be switched. In particular, it should be noted that in all of the examples and figures described herein, the inlets and outlets (and flow direction) may be switched.

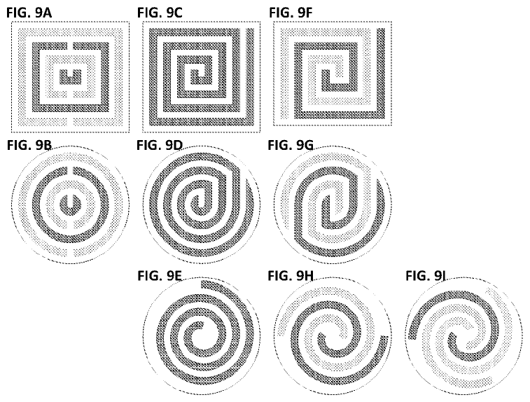

FIG. 9. Many FwB configurations with a distributed outlet region are possible, as shown in examples (FIG. 9A)-(FIG. 9I). These figures should be understood to feature an inlet at the center of the configuration and a distributed outlet region around the periphery (not shown). The filter regions and bypass channels that they define may be rectangular, as in (FIG. 9A), (FIG. 9C), and (FIG. 9F); curved as in (FIG. 9B), (FIG. 9E), (FIG. 9H), and (FIG. 9I); or a combination of both, as in (FIG. 9D) and (FIG. 9G). Other configurations are also possible in addition to these. A configuration may feature multiple separate (or discontinuous) filter regions, as in (FIG. 9A), (FIG. 9B), and (FIG. 9F)-(FIG. 9I) or a single (continuous) filter region that is “wrapped” around the central inlet in a spiral or spiral-like manner, as in (FIG. 9C)-(FIG. 9E). A configuration may feature multiple separate (or discontinuous) filter regions wrapped around the central inlet in a spiral or spiral-like manner, as in (FIG. 9F)-(FIG. 9I).

In these example configurations, the direct flow path is outward (approximately radial) from the central inlet. During operation, the filter region nearest the inlet clogs first. The flow then follows the bypass channels until reaching an unclogged filter region. From there it follows an outward (approximately radial) flow path toward the outlet region. However, this direction may be reversed in other embodiments.

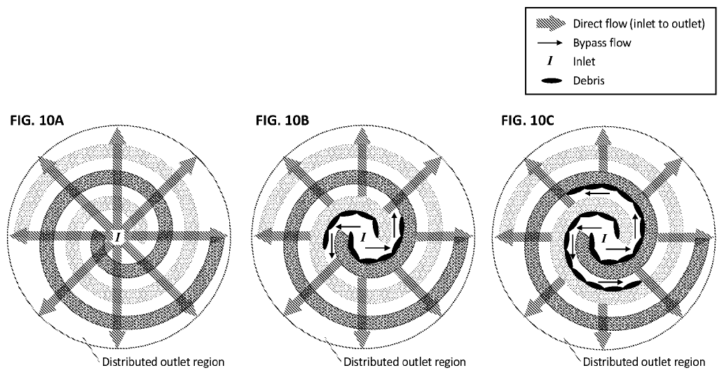

FIG. 10. (FIG. 10A) An FwB configuration with two filter regions that spiral from the central inlet to the distributed outlet region is shown in this example. In the absence of clogging, the flow is predominantly radial with streamlines passing through each filter region one or more times. (FIG. 10B) As debris collects in the central portion of the filter regions, the flow follows a spiral bypass channel until reaching an unclogged portion of the filter region. From there, it follows a predominantly radial flow path toward the distributed outlet region. (FIG. 10C) With additional debris accumulation, the flow follows the bypass channel farther to reach an unclogged portion of the filter region. From there, it follows a predominantly radial flow path toward the distributed outlet region in this example.

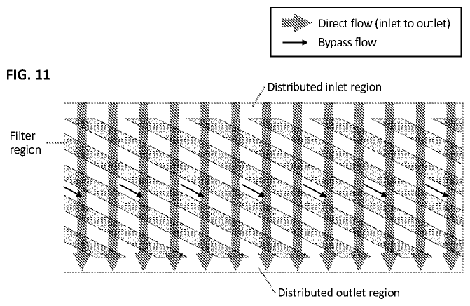

FIG. 11. An FwB configuration may have both a distributed inlet region and distributed outlet region is shown in this example. The predominant flow path is from inlet to outlet in this figure. The filter regions form bypass channels at an angle with respect to the direct flow path. As debris accumulates on the filter regions near the inlet, the bypass channels allow the flow to access the unclogged filter regions further downstream. This diagram may be interpreted as a cross-section of a device that extends into the page or of an approximately planar device with depth into the page greatly exceeded by the outer length and width dimensions.

Accordingly, any of the planar devices described herein may be configured and operated as shown or in some cases, be configured in a rolled configuration with an approximately cylindrical shape. A rolled configuration may have practical advantages in certain embodiments, such as more straightforward macro-micro interfacing and fluid distribution at the inlet and outlet. For example, the planar sheet could be formed and sealed (lidded) using an embossing or roll-to-roll process, tightly rolled around a solid cylinder, and then placed into a tight-fitting cylindrical housing. End caps could then be added to seal the assembly, distribute inlet and outlet fluid flow, and connect to standard tubing.

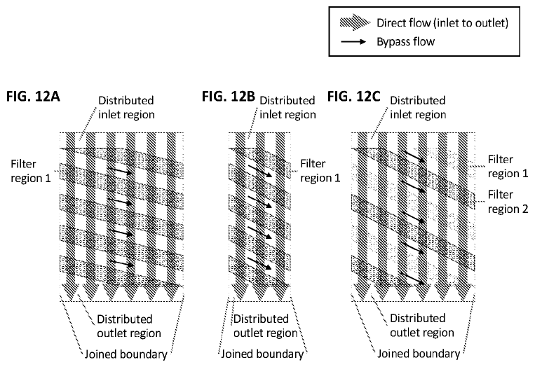

FIG. 12. (FIG. 12A) An FwB configuration formed by wrapping a sheet to form a cylindrical shell is shown in this example. The features on the sheet are periodic such that they match at the joined boundary. The resulting cylindrical filter may have a single, continuous filter region. Thus, for example, in three dimensions, the filter region forms a helix, and the bypass channel also forms a helix. The predominant flow path may be from inlet to outlet. The filter region may form a bypass channel at an angle with respect to the direct flow path. As debris accumulates on the filter region near the inlet, the bypass channel may allow the flow to access the unclogged filter regions further downstream. (FIG. 12B) The fluidic resistance of the bypass channel relative to the filter region may be altered in some embodiments, for example, by changing the length of the bypass channel. In certain embodiments, reducing the width of the sheet while maintaining the periodic boundaries reduces the length of the bypass channel and/or also may reduce the width through which the direct flow passes. These may serve to reduce the fluidic resistance of the bypass channel relative to the filter region. (FIG. 12C) A cylindrical FwB may also use multiple, continuous filter regions in another set of embodiments. In three dimensions, the FwB shown with two filter regions may form a double helix, and/or the bypass channels may form a double helix. The increase in the number of bypass channels relative to the otherwise similar configuration in (FIG. 12A) may reduce the length of each bypass channel and increase their number. This may in some embodiments reduce the total parallel fluidic resistance of the bypass channels relative to the filter regions.

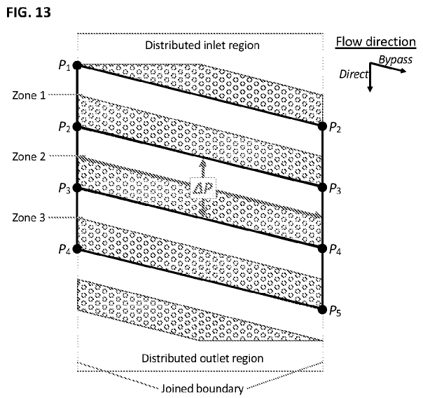

FIG. 13. An FwB may be subdivided into filter zones, or subunits. One example is shown in FIG. 13. This may be useful for describing the characteristics of an FwB on a local level. A filter zone may include a section crossing a filter region for which the pressure drop in the direct flow direction is the same as the pressure drop in the bypass flow direction. Here, it should be understood that the direct flow direction refers to the direction of a shortest path from inlet to outlet. While it is convenient to define a zone with boundaries aligned to the filter region (or bypass channel), as in this figure, it is not a requirement.

The FwB may be divided into any number of filter zones, for example, 2, 3, 4, 5, 6, 7, 8, 9, 10, or more. In the example in FIG. 13, three zones are shown (Zone 1, Zone 2, and Zone 3) for the filter shown in (FIG. 12A). Pressure values P₁, P₂, P₃, P₄, and P₅ are indicated at the corners of the zones, and the left and right edges of the filter are joined. Using Zone 2 as a representative non-limiting example, one procedure for identifying a filter zone is as follows. Zone 2 has a first corner point with pressure P₂. A first boundary of Zone 2 extends from the first corner point (with pressure P₂) in the direct flow direction across a bypass channel and filter region to a second corner point with pressure P₃. A second boundary of Zone 2 extends from the first corner point (with pressure P₂) in the bypass flow direction to a third corner point with pressure P₃. A third boundary of Zone 2 extends from the third corner point (with pressure P₃) across a bypass channel and filter region to a fourth corner point with pressure P₄. Finally, a fourth boundary of Zone 2 extends from the second corner point (with pressure P₃) to the fourth corner point with pressure P₄.

As shown in the figure, in the direct flow direction pressure drops from values in range (P₁, P₂) to values in range (P₂, P₃). In the bypass flow direction pressure also drops from values in the range (P₁, P₂) to values in the range (P₂, P₃). The characteristic pressure drop in both directions is ΔP, as shown in the figure.

The component of flow in the bypass direction is Q_{b}=4P/R_{b}, where R_{b} is the resistance of the filter zone in the bypass flow direction. The component of flow in the direct direction is Q_{d}=4P/R_{d}, where R_{d} is the resistance of the filter zone in the direct flow direction.

Within a filter zone, the local filtration fraction (LFF) may then be defined as the ratio of the direct component of the flow to the sum of the direct and bypass components of flow:

$$LFF = {Q}_{d} / ( {Q}_{d} + {Q}_{b} ) = {R}_{b} / ( {R}_{b} + {R}_{d} ) .$$

The LFF describes the tendency of the fluid to flow through the filter region as opposed to the through the bypass channel. From the equation it can be seen that the LFF approaches 1 when R_{b}>>R_{d}, indicating that flow tends toward the direct path through the filter when the resistance of the bypass is larger than the resistance of the filter region. From the equation, it also can be seen that the LFF approaches 0 when R_{b}<<R_{d}, indicating that flow tends toward the bypass path “around” the filter region when the resistance of the filter region is larger than the resistance of the bypass, e.g., when the filter region becomes clogged with debris.

The LFF may vary across filter zones within an FwB device. The LFF may also change as the filter is used. In some cases, debris accumulation may increase the resistance of the filter region and thereby increase the resistance of the filter zone in the direct flow direction.

The LFF may be used to estimate how many times a flow streamline passes through a filter region in proceeding from the inlet to the outlet, in certain embodiments. The number of filtrations (NF) may be the sum of the LFF values for the contiguous, nonoverlapping zones encountered by a flow proceeding from the inlet to the outlet of the FwB either in the direct flow direction or the bypass flow direction. (The number of zones may be the same for both because the pressure drop in each direction for each zone is the same and the sum of the zone pressure drops equals the total pressure drop from inlet to outlet.) For example, if flow passes through five zones with LFF values of 0.5, 0.6, 0.7, 0.8, and 0.9, then NF=0.5+0.6+0.7+0.8+0.9=3.5. That is, on average flow streamlines pass through a filter region 3.5 times.

The LFF can also be identified in some embodiments as a continuous function of position along a filter region (or bypass channel) from inlet to outlet because the first point of a zone can be defined at any position. With this approach, NF can be found by integrating LFF per zone length in the bypass flow direction from the initial position to final position.

In some embodiments, the NF may be greater than 1, e.g., such that all flow passes through a filter region at least once. Because the LFF (and NF) will tend to decrease as debris accumulates in a filter, it may be desirable in certain cases for NF to be much greater than 1 when the filter is in its initial (unclogged) state. This will allow NF to remain greater than 1 as debris accumulates in the filter. For example, the initial NF may be greater than 1, greater than 2. greater than 5, greater than 10, greater than 20, or greater than 50, etc. Other initial NF values are also possible.

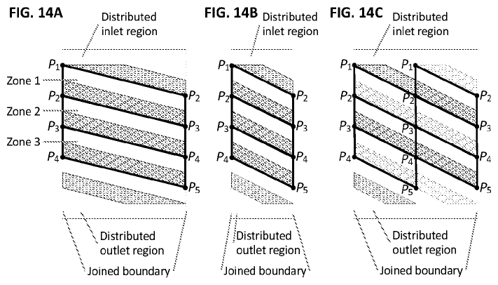

FIG. 14 shows filter zone examples for each of the FwB configurations in FIG. 12. as non-limiting examples. FIG. 14A shows three filter zones for the filter in FIG. 12A following the procedure described for FIG. 13. FIG. 14B shows three filter zones for the filter in FIG. 12B following the procedure described for FIG. 13. FIG. 14C shows six zones for the filter in FIG. 12C. This filter has two separate, rotationally symmetric filter regions. The pressure values at the points shown is apparent from the rotational symmetry of the filter. The zones may then be defined following the procedure described for FIG. 13A.

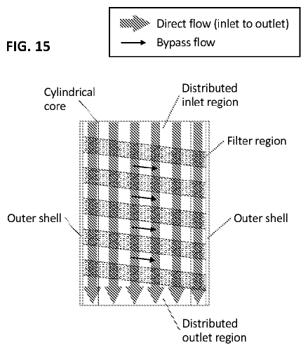

FIG. 15. An FwB configuration formed by winding a filter region around a cylindrical core is shown in this example. The FwB assembly may be prepared by placing the filter and core into a cylindrical outer shell, or by wrapping the filter and core with a laminate, etc. In three dimensions, for example, the filter region may form a helix, and/or the bypass channel may form a helix. The predominant flow path is from inlet to outlet. The filter region may form a bypass channel at an angle with respect to the direct flow path. As debris accumulates on the filter region near the inlet, the bypass channel may allow the flow to access the unclogged filter regions further downstream.

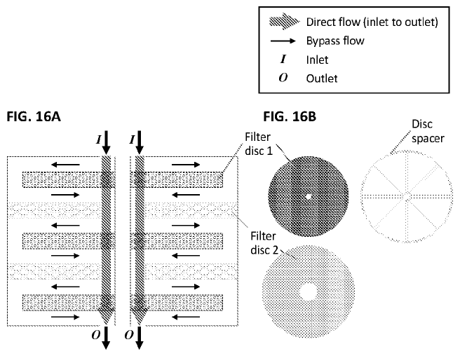

FIG. 16. An FwB may be assembled from planar filter membranes, as this non-limiting example shows. (FIG. 16A) Cross-section of an example cylindrical FwB assembly. Disc-shaped filters of two types may be alternately stacked with spacers (not shown) placed between filter layers. The spaces between the filter disc may thus form a bypass channel. The spacer may minimally impact flow through the filter discs and/or the bypass channel. The direct flow path is from inlet to outlet (perpendicular to the filter disc surface). The bypass flow may be parallel to the filter disc surface except at the edges of the disc where the bypass flow passes around the edge of the filter disc. (FIG. 16B) Top view of key components of the FwB in this particular example. Filter disc 1 has a smaller center-hole than Filter disc 2, and Filter disc 1 has a smaller outer diameter than Filter disc 2.

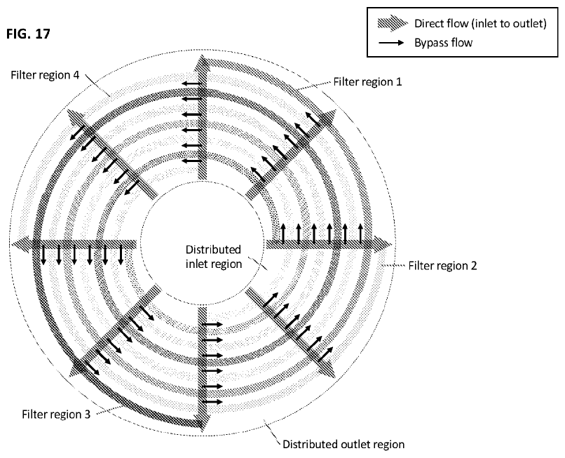

FIG. 17. A microfluidic FwB design with four parallel filter regions that spiral from the distributed inlet region near the hub of the disc to the distributed outlet region near the rim of the disc is shown in this example. The filter regions define and are separated by four parallel bypass channels that also spiral from the distributed inlet region near the hub of the disc to the distributed outlet region near the rim of the disc in this example. In the absence of clogging the flow may be predominantly radial with streamlines passing through each filter region one or more times. Because the fluidic resistance of the bypass (spiral path) may be higher than that of the filter region (radial path), the fluid may increasingly take the bypass as the filter becomes clogged (increasing the filter region resistance). The full filter area on the disc may be utilized because the fluid retains access to and flow paths through unclogged filter regions closer to the rim. For comparison, consider a concentric filter arrangement: when the innermost filter region becomes clogged, the device becomes non-functional, regardless of the amount of available (unclogged) filter area in the outer rim of the device. In addition, as discussed herein, the inlet and outlet can be switched in some embodiments. The flow paths shown would then be reversed.

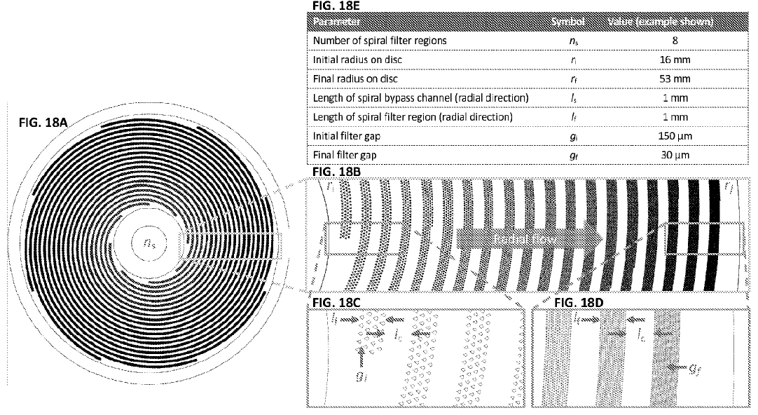

FIG. 18. Design details of a non-limiting example microfluidic FwB design. (FIG. 18A) The design in this example features 8 spiral filter regions, each extending from the distributed inlet region near the disc hub to the distributed outlet region near the disc rim. (FIG. 18B) In the absence of clogging, the flow follows a predominantly radial flow path. The exact path may depend on the fluidic resistance of the filter regions relative to the bypass channels. As shown, the filter regions comprise an array of kite-shaped posts designed to split flow and trap debris. The size and spacing of the posts may vary from the inlet (near the hub) to the outlet (near the rim). As shown, both decrease linearly as a function of radial position on the disc. The filter region properties may be invariant, or vary in other characteristics or according to non-linear functions, in various embodiments. (FIG. 18C) and (FIG. 18D) Close-up view of the microfluidic features near the (FIG. 18C) disc hub and (FIG. 18D) disc rim. The features and parameters called out in (FIG. 18C) and (FIG. 18D) may be defined in the table in (FIG. 18E) along with the values for the design shown, as one non-limiting example. Many other sets of parameters are possible. A practical advantage of certain spiral FwB designs, like the one shown, is that their rotational symmetry and smoothly varying design properties facilitate algorithmic design generation.

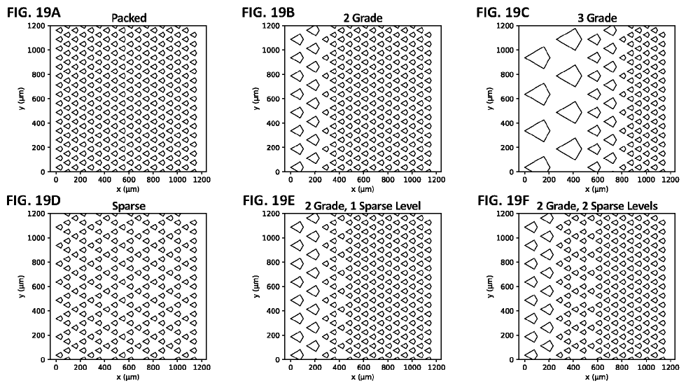

FIG. 19 illustrates various non-limiting microfluidic filter region examples. Each example in FIG. 19 shows a portion of a filter region that may be extended in any direction along a linear, curvilinear, spiral, or any other geometric path. In these examples, the flow proceeds in a direction that is generally from left to right, though other directions are also possible. The array of kite-shaped posts repeatedly splits flow through the array along varying streamlines (although it should be understood that the kite-shaped posts are presented here by way of example only, and other shapes are also possible in other embodiments, e.g., as described herein). This may increase the likelihood that elongated and fibrous debris will span streamlines flowing around opposite sides of a post and thereby wrap around the post and become trapped.

Variations of the array pattern, post size, post shape, post size, and post spacing (gap between posts), and post height, among other properties, may be used to alter or enhance the effectiveness of filtration and capacity of the filter region, in various embodiments. These properties may be constant or may vary throughout an FwB. It should be understood that, in various embodiments, the array characteristics may influence the fluidic resistance of the filter region and may affect, for example, the LFF, NF, and/or other overall filter performance characteristics. The non-limiting examples shown here demonstrate just a small number of many possible filter region designs envisioned for FwB devices in accordance with various embodiments described herein.

FIG. 19A illustrates an array of posts with a single size grade. FIG. 19B illustrates an array of posts with two size grades. The larger initial (upstream) post spacing may trap larger debris while allowing smaller debris to penetrate deeper into the filter region before getting trapped, thereby increasing filter capacity. FIG. 19C illustrates an array of posts with three size grades. As in FIG. 19B, the larger initial (upstream) post spacing may trap larger debris while allowing smaller debris to penetrate deeper into the filter region before getting trapped, thereby increasing filter capacity. FIG. 19D illustrates an array of posts with a single size grade. This array is a variant of the array shown in FIG. 19A with posts selectively removed to create pockets of space (sparsity). This additional space may allow for increased debris accumulation, thereby increasing filter capacity. FIG. 19E illustrates a mixed array of posts with two size grades. One level of sparsity is added to the array shown in FIG. 19B with the remainder of the array remaining dense. The combination of multiple grades and sparsity may provide the aforementioned benefits of both array sparsity and grading. FIG. 19F illustrates a mixed array of posts with three size grades. Two levels of sparsity are added to the array shown in FIG. 19B with the remainder of the array remaining dense. The combination of multiple grades and sparsity may provide the aforementioned benefits of both array sparsity and grading.

Thus, it will be understood that in various embodiments, a variety of different obstacles and posts can be used, including having the same or different shapes and/or sizes of posts within a filter, different spatial arrangements of posts within a filter, etc. For example, posts may have shapes such as square, triangular, rectangular, circular, kite-shaped, irregular, etc., and the posts may be regularly or irregularly positioned in an array within a filter. In some cases, the posts may be positioned in a regular array with some members of the array missing, e.g., to create space to contain debris. Other configurations are also possible in yet other embodiments.

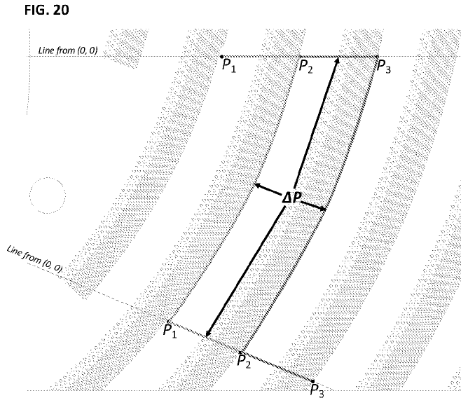

FIG. 20. Non-limiting example of a filter zone in a spiral FwB device with multiple filter regions. The spirals defining the filter regions and bypass channels between them are centered at the origin (0, 0). Lines from the origin through the initial position of each filter region indicate the rotational symmetry of the filter. Pressure values P₁, P₂, and P₃ are shown on each of these lines at equivalent (by symmetry) positions. The direct flow direction is radial, e.g., outward along each of the lines from the origin. The bypass flow direction follows the bypass channel along a spiral path.

The filter zone shown in this non-limiting example has a first corner point with pressure P₁. A first boundary extends from the first corner point (with pressure P₁) in the direct flow direction across a bypass channel and filter region to a second corner point with pressure P₂. A second boundary of extends from the first corner point (with pressure P₁) in the bypass flow direction to a third corner point with pressure P₂. A third boundary extends from the third corner point (with pressure P₂) across a bypass channel and filter region to a fourth corner point with pressure P₃. Finally, a fourth boundary extends from the second corner point (with pressure P₂) to the fourth corner point with pressure P₃.

As is apparent in the figure, in the direct flow direction pressure drops from values in range (P₁, P₂) to values in range (P₂, P₃). In the bypass flow direction pressure also drops from values in the range (P₁, P₂) to values in the range (P₂, P₃). The characteristic pressure drop in both directions is AP, as shown in the figure.

As described previously, the LFF may be understood to be the ratio of the direct component of the flow to the sum of the direct and bypass components of flow and is calculated as LFF=R_{b}/(R_{b}+R_{d}), where R_{b} is the resistance of the filter zone in the bypass flow direction and R_{d} is the resistance of the filter zone in the direct flow direction.

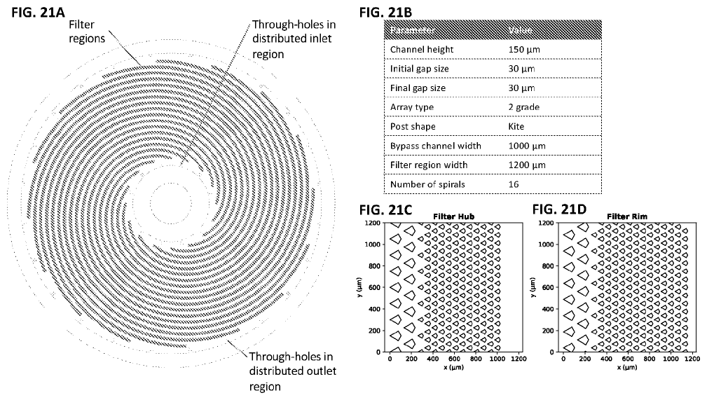

FIG. 21. Design details of a non-limiting example microfluidic FwB design. FIG. 21A shows an example of a device in disc format with 120 mm outer diameter. Inlet through-holes distribute fluid near the disc hub, and outlet through-holes collect fluid near the disc rim. The design features 16 rotationally symmetric filter regions and bypass channels extending from the distributed inlet region to the distributed outlet region. FIG. 21B shows certain characteristics of the design are summarized in the table shown. FIG. 21C and FIG. 21D show the filter region layout and dimensions near the disc hub (inlet region) and disc rim (outlet region). The array of posts is dense and has two grades. The width of the filter region (in the direct flow direction) varies from about 1,000 micrometers near the inlet to about 1,200 micrometers near the outlet. However, it should be understood that this is by way of example only, and that in other embodiments, other filter arrangements, outlets, symmetrics, etc. are also possible, e.g., including any of those described herein.

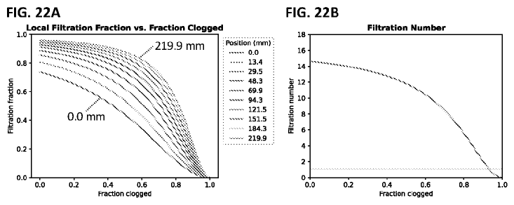

FIG. 22. Filtration characteristics of the non-limiting example microfluidic FwB device in FIG. 21. FIG. 22A shows that for the device shown in FIG. 21, the local filtration fraction (LFF) was calculated as a function of position along a filter region from inlet (position=0.0 mm) to outlet (position=219.9 mm) for varying levels of filter clogging. Here the fraction clogged is the fraction of the width between posts in the filter region that is occupied by debris, effectively reducing the width between posts. As shown in the plot, for any given value of clogging the LFF increases from inlet to outlet. This is because in proceeding from the inlet to the outlet of the disc the filter zones become increasingly long in the bypass flow direction and increasingly wide the direct flow direction. As a result, the resistance in the bypass flow direction increases and the resistance in the direct flow direction decreases. Both contribute to increasing the LFF from inlet to outlet because LFF=R_{b}/(R_{b}+R_{d})), where R_{b} is the resistance of the filter zone in the bypass flow direction and R_{d} is the resistance of the filter zone in the direct flow direction. As the fraction clogged increases, the resistance in the direct flow direction increases and causes the LFF to decrease. As the fraction clogged approaches 1, the LFF approaches 0, indicating that most flow passes through the bypass channels. (FIG. 22B) The number of filtrations (NF) (“filtration number” on the plot) is calculated through integration of the LFF as described previously. NF describes the number of times flow streamlines pass through filter regions in proceeding from the inlet to the out. For the example device described here, the fluid passes through about 14 filter regions when then the filter is unclogged (fraction clogged=0). NF gradually drops to about 12 filter regions when the filter is 50% clogged (fraction clogged=0.5), and then begins a sharper decline. NF does not fall below 1 until the filter is >90% clogged (fraction clogged>0.9). Thus, the fluid continues to be filtered until the FwB is almost entirely clogged with debris. In contrast to conventional filters, fluid will continue to flow (via the bypass channels) when the FwB reaches this point, enabling other processing modules in series with the filter to continue to operate.

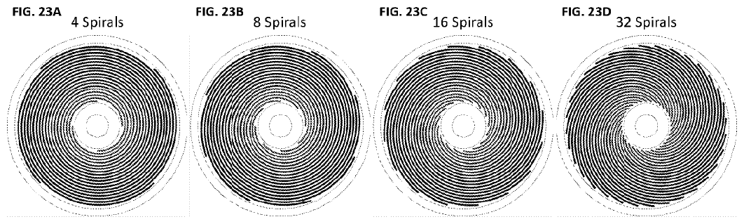

FIG. 23. Microfluidic FwB designs with (FIG. 23A) 4 spiral filter regions and bypass channels, (FIG. 23B) 8 spiral filter regions and bypass channels, (FIG. 23C) 16 spiral filter regions and bypass channels, and (FIG. 23D) 32 spiral filter regions and bypass channels, are shown as additional non-limiting examples. The number of spirals may determine the relative resistance between filter regions and the bypass channels. The total parallel resistance of the bypass channels may be related to the inverse square of the number of bypass channels in some embodiments. For instance, increasing the number of spiral bypass channels by a factor f may increase the number parallel paths by f and/or may reduce the length of each path by about 1/f. The combined effect may be to scale the total bypass resistance by 1/f². Given other filter properties, in certain embodiments, the number of spiral regions can be selected to allow balanced, progressive clogging of filter regions from inlet to outlet.

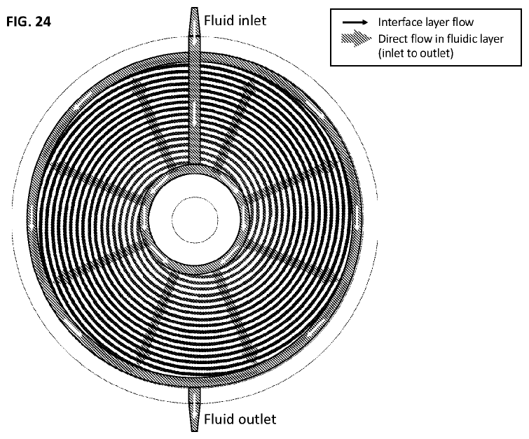

FIG. 24. Interface layer for a spiral microfluidic FwB design, in yet another non-limiting example. The microfluidic device (sans interface layer) in this particular example comprises three layers: base layer, fluidic layer (or filter layer), and lid layer. Either the base layer or lid layer may feature through-holes that allow flow into and out of the microfluidic channels in the fluidic layer. An interface layer may be bonded to the microfluidic device to distribute fluid flow and connect to tubing or other fluidic conduits or modules. In the example device shown, the microfluidic device lid layer features inlet through-holes distributed around the disc hub and outlet through-holes distributed around the disc rim. The interface layer features a channel that directs inlet fluid into the inlet through-holes and a second channel that collect fluid from the outlet through-holes and directs it to the outlet.

The above discussion described various non-limiting example of certain embodiments of the present disclosure. However, other embodiments are also possible. Accordingly, more generally, various aspects of the disclosure are directed to various systems and methods for filtration.

For instance, a variety of materials and methods, according to certain aspects, can be used to form devices or components such as those described herein, e.g., microfluidic devices, etc. For example, various devices or components can be formed from solid materials, in which the channels can be formed via machining or micromachining, 3D-printing, film deposition processes such as spin coating and chemical vapor deposition, physical vapor deposition, laser fabrication, photolithographic techniques, etching methods including wet chemical or plasma processes, electrodeposition, 3D-printing, hot embossing, injection molding, lamination, laser cutting, soft lithography, or the like.

In one set of embodiments, various structures or components of the devices described herein can be formed of materials such as glass, metals, polymers, etc. For example, the device may be formed from an elastomeric polymer such as polydimethylsiloxane (“PDMS”). polytetrafluoroethylene (“PTFE”), or the like. Other examples of potentially suitable polymers include, but are not limited to, polyethylene terephthalate (PET), polyacrylate, polymethacrylate, polycarbonate, polystyrene, polyethylene, polypropylene, polyvinylchloride, cyclic olefin copolymer (COC), polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, or the like. Still other materials include, but are not limited to, PDMS, glass, silicon, ceramic, polymer, elastomer, COC (cyclic olefin copolymer), COP (cyclic olefin polymer), PMMA (polymethyl methacrylate), nylon, polypropylene, polyethylene, polyester, polystyrene, PTFE, cellulose, cellulose acetate, carbon fiber, glass fiber, or stainless steel. Combinations, copolymers, or blends involving polymers including those described above are also envisioned. The device may also be formed from composite materials, for example, a composite of a polymer and a semiconductor material.

In some embodiments, the filter region may be constructed of PDMS, glass, silicon, ceramic, polymer, elastomer, COC, COP, PMMA, nylon, polypropylene, polyethylene, polyester, polystyrene, PTFE, cellulose, cellulose acetate, carbon fiber, glass fiber, stainless steel, and/or other materials, including any of those described herein. The base, lid, and other parts of the filter assembly may be constructed PDMS, glass, silicon, ceramic, polymer, elastomer, COC, COP, PMMA, nylon, polypropylene, polyethylene, polyester, polystyrene, PTFE, cellulose, stainless steel, aluminum, adhesive, and/or other materials.

The filter components, including the filter region, base, lid, and other parts of the filter assembly, if present, may be fabricated using any suitable technique, such as casting, molding, embossing, extrusion, photolithography, machining, micromachining, sintering, spinning, weaving, packing, lamination, printing, injection molding, and/or other microfabrication and macrofabrication processes, including any of those described herein.

The filter components may be modified in some embodiments to introduce holes, pores, fluidic channels, or to provide structural support. They may be sterilized, for example, using gamma irradiation, electron beam irradiation, high temperature, chemical treatment, and/or other techniques.

The materials may also be treated to change their wettability or contact angle. The materials may be treated to change or improve their compatibility with biological and chemical substances. The materials may be treated to change their affinity for debris or other substances in either a specific or non-specific manner. For example, the filter may be coated with antibody targeting an antigen expressed on a particular type of cell or debris. The filter may also be coated with a chemical or protein that facilitates subsequent modification. For example, the filter may be coated with biotin to enable subsequent binding to avidin on an antibody, cell, bead, debris, or other analyte.

An example process for fabricating a microfluidic device according to one embodiment of the present disclosure is set forth as follows. However, other techniques for making microfluidic devices will be known to those of ordinary skill in the art.

In this example, a substrate layer is first provided. The substrate layer can include, e.g., glass, plastic, or silicon wafer. An optional thin film layer (e.g., SiO₂) can be formed on a surface of the substrate layer using, for example, thermal or electron beam deposition. The substrate and optional thin film layer provide a base in which the microfluidic channels can be formed. The thickness of the substrate can fall within the range of approximately 500 micrometers to approximately 10 mm. For example, the thickness of the substrate layer can be at least 600 micrometers, at least 750 micrometers, at least 900 micrometers, at least 1 mm, at least 2 mm, at least 3 mm, at least 4 mm, at least 5 mm, at least 6 mm, at least 7 mm, at least 8 mm, at least or 9 mm. Other thicknesses are possible as well.

The microfluidic channels formed within the substrate may include the different fluid flow pathways for a fluid, such as the straight channels, filters (including those described herein), fluidic resistors, etc. The microfluidic channels can be formed, in some implementations, by depositing a polymer (e.g., polydimethylsiloxane (PDMS), polymethylmethacrylate (PMMA), polycarbonate (PC), or cyclo olefin polymer (COP)) in a mold that defines the fluidic channel regions. The polymer, once cured, then can be transferred and bonded to a surface of a support layer. For example, PDMS can be first poured into a mold (e.g., an SU-8 mold fabricated with two step photolithography (MicroChem)) that defines the microfluidic network of channels. The PDMS then is cured (e.g., heating at least 65° C. for about 3 hours). Prior to transferring the solid PDMS structure to the support layer, the surface of the substrate layer may be treated with O₂ plasma to enhance bonding. In some embodiments, if the microfluidic channels are fabricated in other substrate materials, such as a glass or silicon wafer, the channels can be formed using standard semiconductor photolithography processing to define the channel regions in combination with wet and/or dry etching techniques to fabricate the channels. Additional non-limiting examples of microfluidic channel networks and their fabrication can be found. for example, in U.S. Pat. Apl. Nos. 2020/0139370 or 2011/0091987, or U.S. Pat. Nos. 8,021,614, and 8,186,913.

The characteristics of the filter regions may vary from one region to another region. They may also vary within a single, continuous filter region. The materials, dimensions, and design details (e.g., shape, size, spacing) may all vary. Examples include any of those described herein, e.g., with reference to the figures. Filter regions may be graded, with the feature spacing varying from one region to another. If coatings or surface modification are used, they may also vary from one region to another.

The characteristics of the bypass channels may vary across a device. The bypass channel dimensions may vary spatially. The bypass channel may also open, move, deform, or change as a function of fluid pressure, fluid flow rate, or external actuation. For example, a pressure increase due filter clogging may induce the adjacent bypass channel to open or expand, increasing bypass flow. The bypass channel may also incorporate ridges and/or other features to prevent debris that accumulates on a filter region from rolling or sliding along the bypass channel.

Fluids may be pumped through the filter using one or more of many techniques. These techniques include gravity, positive pressure (on inlet side), negative pressure (vacuum on outlet side), peristaltic pump, centrifugal force, and positive displacement as in a syringe pump or bag under compression. Valves may be used to control flow into the inlet and/or out of the outlet.

Prior to sample filtration, a priming process may be used in some embodiments to remove air from the filter. This process may use a fluid with better wetting characteristics than those of the sample. For example, the priming fluid may contain an alcohol, protein, or surfactant. The priming process may also use fluid flow or mechanical agitation to free bubbles from surfaces.

Prior to priming, in some embodiments, a vacuum may be used to evacuate gas (air) from the filter and thereby minimize the amount of gas that may be trapped during priming.

The filter may be directly integrated with another filter or module fabricated in the same (or different) substrate, housing, or assembly. It may also be integrated with another filter or module directly connected (e.g., by welding or adhesion) to its substrate, housing, or assembly.

The filter may be integrated with other filters or modules via tubing, connectors (e.g., luers, spikes, tube welding, sterile connectors), or other microfluidic or macrofluidic conduits or structures. The integration may be permanent (e.g., welded, solvent-bonded, mechanically locked) or non-permanent (e.g., luers or mechanical disconnection).

The filter may be integrated with almost any equipment incorporating fluid flow in a clinical, biomedical, research, bioprocessing, or manufacturing setting.

The filter may be integrated with other filters in series and/or in parallel.

The filter may be integrated with a with a cell sorting or cell concentration device. Such devices may be microfluidic or macrofluidic, label-based or label-free, active or passive. Devices may use magnetic separation (MACS), FACS, centrifugation, counterflow centrifugation, elutriation, acoustophoresis, inertial focusing, inertial sorting, inertial concentration, deterministic lateral displacement, spinning membrane filtration, flow sorting, and/or droplet sorting. Other techniques may also be used.

The filter may be integrated with a blood processing device or other medical device. Examples include equipment for apheresis, photopheresis, infusion, heart-lung bypass, dialysis, centrifugation, autotransfusion, and transfusion.

The filter may be integrated with a diagnostic or analytical device, such as a flow cytometer, hematology analyzer, coulter counter, microscope, or DNA sequencer.

The filter may be integrated with a fluid handling system, aliquoting system, mixer, valve, incubator, refrigerator, or fluid storage system. The filter may be integrated with sensors that measure flow rate, pressure, filter clogging, or other process information. Such process information and feedback may be logged for later analysis or used to for real-time process adjustments.

The filter may be integrated with equipment used for bioprocessing, biomanufacturing, cell banking, and cell and gene therapy research and manufacturing. These include equipment for thawing, freezing, thawing, warming, and washing samples, as well as equipment for transfecting and/or transducing cells, such as electroporation, mechanoporation, and viral and nanoparticle delivery systems. These also include bioreactors, fermenters, cell culture systems, chromatography systems, filtration and clarification systems, fill and finish systems, analytical tools, and quality control tools.

Filters including those described herein can be used in various different applications. For example, the techniques and devices disclosed herein can be used to filter fluid samples in some embodiments. Such fluids can include, e.g., blood, aqueous solutions, oils, or gases. In some embodiments, the techniques and devices disclosed herein can be used to isolate and enrich or purify fluid samples, for example, containing cells or other biological samples. Such cells can include, e.g., blood cells in general as well as fetal blood cells in maternal blood, bone marrow cells, and circulating tumor cells (CTCs), sperm, eggs, bacteria, fungi, virus, algae, any prokaryotic or eukaryotic cells. Fluid samples can also include organelles, exosomes, microvesicles, droplets, bubbles, pollutants, precipitates, clumps, aggregates, beads, gels, hydrogels, fibers, organic and inorganic particles, magnetic beads, and/or magnetically labeled analytes), or the like.

The filter may be used to filter many different types of fluids. These include peripheral blood, apheresis, leukapheresis, plasma, red blood cells, platelets, cord blood, other blood products, bone marrow aspirate, adipose-derived fluid, lavage, urine, sputum, semen, and other body fluids. The fluid may be based on saline, buffer, culture media, plasma, cryoprotectant, alcohol, oil, and/or other fluids. The fluid may include substances that modify its density, viscosity, color, pH, acoustic characteristics, electrical characteristics, or other properties. The fluid may also contain electrolytes, proteins, surfactants, anticoagulants, DMSO, DNase, sugars, enzymes, antibodies, beads, labels, and/or reagents.

The sample fluid may contain eukaryotic cells, prokaryotic cells, blood cells, red blood cells, platelets, leukocytes, granulocytes, monocytes, lymphocytes, T cells, NK cells, stem cells, cancer cells, yeast, bacteria, cultured cells, rare cells, cell clusters, cell clumps, live cells, dead cells, lysed cells, intracellular material, organelles, plasma, clotting factors, clots, antibodies, DNA, RNA, cellular debris, droplets, beads, nanoparticles, and/or microcarriers.

The retentate (material retained by the filter) may include eukaryotic cells, prokaryotic cells, blood cells, red blood cells, platelets, leukocytes, granulocytes, monocytes, lymphocytes, T cells, NK cells, stem cells, cancer cells, yeast, bacteria, cultured cells, rare cells, cell clusters, cell clumps, live cells, dead cells, lysed cells, intracellular material, organelles, plasma, clotting factors, clots, antibodies, DNA, RNA, cellular debris, droplets, beads, nanoparticles, and/or microcarriers.

Following the filtration process, the filter and its contents may be analyzed. The filter maybe constructed to allow it to open, providing direct access to the retentate. The filter may be designed to enable extraction of the retentate by retrograde flow or by adding an enzyme or other reagent to free the material from the filter surface. The retentate may be labeled (e.g., fluorophore-conjugated antibodies), lysed, or otherwise modified, to facilitate analysis or other usage.

In some applications the purpose of the filter may be to remove debris or other unwanted materials from the filtrate. In other applications, the purpose of the filter may be to retain wanted material in the retentate. This may include a particular type of cell (e.g., circulating tumor cells, cell clusters, stem cells, T cells, NK cells, microorganisms), biological material (e.g., cell lysate, organelles, clots, antibodies, protein, lipid, DNA, RNA, virus), or other type of material (e.g., droplets, beads, microcarriers, nanoparticles, analytes) of diagnostic, therapeutic, or research interest. The filter may target cell surface antigens, biophysical characteristics (e.g., size), or other properties. The filter features may use wells that enable cells or other fluid components to be collected during figuration and subsequently recovered.

U.S. Provisional Patent Application Ser. No. 63/346,701, filed May 27, 2022, entitled “Systems and Methods for Filtration,” is incorporated herein by reference in its entirety.

The following examples are intended to illustrate certain embodiments of the present disclosure, but do not exemplify the full scope of the disclosure.

### EXAMPLE 1

Autologous cell therapy is a groundbreaking approach to treating cancer and other diseases by isolating, genetically engineering, expanding patient-derived blood cells, and then reinfusing the cells into the patient where they seek out and destroy cancer cells. Unfortunately, scaling cell therapies has been hampered by manufacturing challenges that are driven in part by sample variability and its effect on manufacturing processes, analytics, and cost.

The input to most the cell therapy manufacturing processes is leukapheresis. Leukapheresis is a patient-derived blood product that typically has 200-500 mL volume, ≥10 billion nucleated cells, ≥10 billion red blood cells, and hundreds of billions of platelets. Post-collection, the sample is stored/shipped for up to 48 hours and then either enters the therapy manufacturing process or an interim process to enable cryopreservation and freezing. Tremendous variability is introduced by factors including the patient, pre-treatment (e.g., chemotherapy), apheresis process conditions, storage conditions, sample age (and attendant cell death) at the start of manufacturing, and the cryopreservation process. Post-thaw the sample typically includes dead cells (granulocytes in particular), cell debris, cell clumps, free DNA, lysed red blood cells, and platelet clumps. These contaminants complicate and interfere with subsequent process steps, such as cell washing, cell sorting, cell activation, and cell expansion. While some steps are taken to address contaminants, including filtration and treatment with DNAse, more effective techniques are needed.

This example provides a more effective system of removing debris from leukapheresis samples and facilitate more consistent, higher performance manufacturing processes for cell therapies.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or.” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of.” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A device, comprising:
a filter comprising a filter element and a bypass pathway positioned to flow a fluid around the filter element.

2. The device of claim 1, wherein the bypass pathway has a fluidic resistance greater than a fluidic resistance of the filter element.

3. The device of any one of claim 1 or 2, wherein the bypass pathway is a microfluidic channel.

4. The device of any one of claims 1-3, wherein the bypass pathway has a length that is at least 3 times greater than a fluid flow path through the filter element.

5. The device of any one of claims 1-4, wherein the bypass pathway has a length that is at least 10 times greater than a fluid flow path through the filter element.

6. The device of any one of claims 1-5, comprising a plurality of filter elements, wherein the bypass pathway is positioned to flow a fluid around the plurality of filter elements.

7. The device of any one of claims 1-6, further comprising a second filter element and a second bypass pathway positioned to flow the fluid around the second filter element.

8. The device of any one of claims 1-7, wherein the filter element defines a spiral.

9. The device of any one of claims 1-8, wherein the filter element has an average pore size of less than 250 micrometers.

10. The device of any one of claims 1-9, wherein the filter element has an average pore size of less than 150 micrometers.

11. The device of any one of claims 1-10, wherein the filter element has an average pore size of less than 25 micrometers.

12. The device of any one of claims 1-11, wherein the filter element has an average pore size of less than 1 micrometer.

13. A device, comprising:
a plurality of filter elements defining arms of a spiral, wherein the plurality of filter elements is positioned to define fluid channels between the arms of the spiral.

14. The device of claim 13, wherein the plurality of filter elements have an average pore size of less than 250 micrometers.

15. The device of any one of claim 13 or 14, wherein the plurality of filter elements have an average pore size of less than 150 micrometers.

16. The device of any one of claims 13-15, wherein the plurality of filter elements have an average pore size of less than 25 micrometers.

17. The device of any one of claims 13-16, wherein the device comprises at least 2 arms.

18. The device of any one of claims 13-17, wherein the device comprises at least 3 arms.

19. The device of any one of claims 13-18, wherein the device comprises at least 5 arms.

20. The device of any one of claims 13-19, wherein the fluid channels have a fluid resistance greater than a fluid resistance of the plurality of filter elements.

21. The device of any one of claims 13-20, wherein the fluid channels are microfluidic channels.

22. The device of any one of claims 13-21, wherein the fluid channels have a length that is at least 10 times greater than a fluid flow path through the filter element.

23. A device, comprising:
a plurality of symmetric filter elements extending from an inlet to an outlet, wherein at least some of the plurality of filter elements are positioned to define fluid channels extending from the inlet to the outlet between the filter elements.

24. The device of claim 23, wherein at least some of the filter elements define spirals.

25. The device of any one of claim 23 or 24, wherein the at least some of filter elements define helices.

26. The device of any one of claims 23-25, wherein the at least some of filter elements define lines.

27. The device of any one of claims 23-26, wherein the at least some of filter elements define curves.

28. The device of any one of claims 23-27, wherein the plurality of filter elements have an average pore size of less than 250 micrometers.

29. The device of any one of claims 23-28, wherein the plurality of filter elements have an average pore size of less than 150 micrometers.

30. The device of any one of claims 23-29, wherein the plurality of filter elements have an average pore size of less than 25 micrometers.

31. The device of any one of claims 23-30, wherein the device comprises at least 2 filter elements.

32. The device of any one of claims 23-31, wherein the device comprises at least 4 filter elements.

33. The device of any one of claims 23-32, wherein the device comprises at least 8 filter elements.

34. The device of any one of claims 23-33, wherein the device comprises at least 16 filter elements.

35. The device of any one of claims 23-34, wherein the device comprises at least 32 filter elements.

36. The device of any one of claims 23-35, wherein the fluid channels have a fluidic resistance greater than a fluidic resistance of the plurality of filter elements in a direction of fluid flow.

37. The device of any one of claims 23-36, wherein the fluid channels are microfluidic channels.

38. The device of any one of claims 23-37, wherein the fluid channels have a length that is at least 10 times greater than a fluid flow path through the filter element.

39. A method, comprising:
providing a device comprising a filter element and a bypass pathway positioned to flow a fluid around the filter element;
flowing a fluid containing debris through the device such that at least some of the fluid flows through the filter element and at least some of the debris becomes entrapped in the filter element; and
subsequently, flowing at least some of the fluid in the bypass pathway around the filter element.

40. The method of claim 39, comprising flowing a fluid containing debris through the filter element such that the filter element increases in fluidic resistance until the fluidic resistance is greater than a fluidic resistance of the fluid in the bypass pathway.

41. The method of any one of claim 39 or 40, comprising flowing the fluid containing debris through the filter element such that a portion of the filter element increases in fluidic resistance at least 2-fold.

42. The method of any one of claims 39-41, comprising flowing the fluid containing debris through the filter element such that a portion of the filter element increases in fluidic resistance at least 4-fold.

43. The method of any one of claims 39-42, comprising flowing the fluid containing debris through the filter element such that a portion of the filter element increases in fluidic resistance at least 10-fold.

44. The method of any one of claims 39-43, comprising flowing the fluid containing debris through the device such that the flow through a portion of the bypass pathway increases at least 2-fold.

45. The method of any one of claims 39-44, comprising flowing the fluid containing debris through the device such that the flow through a portion of the bypass pathway increases at least 4-fold.

46. The method of any one of claims 39-45, comprising flowing the fluid containing debris through the device such that the flow through a portion of the bypass pathway increases at least 10-fold.

47. The method of any one of claims 39-46, wherein the debris comprises cells.

48. The method of any one of claims 39-47, comprising subsequently flowing at least 30 vol % of the fluid in the bypass pathway around the filter element.

49. The method of any one of claims 39-48, comprising subsequently flowing at least 50 vol % of the fluid in the bypass pathway around the filter element.

50. The method of any one of claims 39-49, comprising subsequently flowing at least 80 vol % of the fluid in the bypass pathway around the filter element.

51. The method of any one of claims 39-50, comprising subsequently flowing at least 90 vol % of the fluid in the bypass pathway around the filter element.

52. The method of any one of claims 39-51, wherein the filter element has an average pore size of less than 25 micrometers.

53. The method of any one of claims 39-52, wherein the bypass pathway has a fluidic resistance greater than a fluidic resistance of the filter element prior to flowing the fluid.

54. The method of any one of claims 39-53, wherein the bypass pathway is a microfluidic channel.

55. The method of any one of claims 39-54, wherein the bypass pathway has a length that is at least 3 times greater than a fluid flow path through the filter element.
